(19) United States
(12) Patent Application Publication
(10) Pub. No.: US 2021/0313022 A1
(43) Pub. Date: Oct. 7, 2021

(54) ENGINE FOR AUGMENTED MEDICAL CODING

(71) Applicant: Diagnoss Inc., Pomona, CA (US)

(72) Inventor: Abboud Chaballout, Glendora, CA (US)

(21) Appl. No.: 17/214,606

(22) Filed: Mar. 26, 2021

Related U.S. Application Data
(60) Provisional application No. 63/001,039, filed on Mar. 27, 2020.
(60) Provisional application No. 63/161,327, filed on Mar. 15, 2021.
(60) Provisional application No. 63/161,332, filed on Mar. 15, 2021.
(60) Provisional application No. 63/149,067, filed on Feb. 12, 2021.

(51) Int. Cl. G16H 10/60; G16H 40/67; G16H 20/00; G16H 80/00; G06F 40/205; G16H 50/20; G06F 21/31; G06Q 20/08

(52) U.S. Cl. CPC G16H 10/60; G16H 40/67; G16H 20/00; G06Q 20/0855; G06F 40/205; G16H 50/20; G06F 21/31; G16H 80/00

(57) ABSTRACT

A method for real-time, augmented coding classification of text inputs is provided. The method includes receiving a string input from a client device, the string input being part of a technical chart for a user of the client device, wherein the user is associated with a healthcare provider and the string input comprises a healthcare report of a patient. The method also includes standardizing the string input according to a set of technical rules, storing the string input in a database, and providing at least a portion of the string input to a processing circuit. The method also includes receiving, from the processing circuit, a code associated with the string input, causing a display in the client device to display the code for the user and providing, to the remote client, a result associated with the technical input data. A system to perform the above method is also provided.

### CROSS-REFERENCE TO RELATED APPLICATIONS

The present disclosure is related and claims priority to U.S. Provisional Patent Application No. 63/001,039; entitled ENGINE FOR AUGMENTED MEDICAL CODING to Abboud Chaballout, filed on Mar. 27, 2020; to U.S. Provisional Patent Application No. 63/149,067, entitled ARTIFICIAL INTELLIGENCE IN HEALTHCARE APPLICATIONS, to Abboud Chaballout, filed on Feb. 12, 2021; to U.S. Provisional Patent Application No. 63/161,327, entitled ENGINE FOR AUTO-TRAINING CUSTOM MODELS, to Markus Krause et al., filed on Mar. 15, 2021; and to U.S. Provisional Patent Application No. 63/161,332, entitled GENERALIZED SIDE-BAR SYSTEM FOR AN ELECTRONIC HEALTH RECORD PLATFORM, to Abboud Chaballout, filed on Mar. 15, 2021; the contents of which applications are hereby incorporated by reference in their entirety, for all purposes.

### BACKGROUND

### Field

The present disclosure is related to automated feedback for text input in the context of a computer network. More specifically, embodiments as disclosed herein provide medical coding assignments to text input reporting a patient's medical record.

### Description of Prior Art

In the field of healthcare management, medical coding is an accepted standard for assessing procedures, treatments, and costs. A specific code listing for medical conditions, diagnostics, and procedures is well established. For example, two coding standards are used in conjunction with outpatient settings: the International Statistical Classification of Diseases (ICD) and the Current Procedure Terminology (CPT). A subset of the CPT coding standard includes evaluation and management (E/M) codes. On the other hand, medical records and reports are usually filled in by healthcare personnel in a clinical environment, or sometimes even by the patients themselves (e.g., reporting medication intake, symptoms, and the like), and are subject to personal biases and even language biases from the person preparing the report. Current technology to associate medical coding (e.g., ICD and CPT codes) to medical records relies heavily on human interpretation, sometimes filtered through some basic logic guidance. However, these approaches often miss the subjacent biases of the person writing the medical report, or even the specific clinical conditions in which the report is prepared. Misclassification of medical codes leads to inefficient cost assessment, economic loss, and potentially even erroneous medical practice with deleterious outcome for the patient, the clinic, and the insurance provider.

### SUMMARY

In a first embodiment, a computer-implemented method includes receiving a string input from a client device, the string input being part of a technical chart for a user of the client device. The user is associated with a healthcare provider and the string input comprises a healthcare report of a patient. The computer-implemented method also includes standardizing a portion of, or all of the string input according to a set of technical rules, storing the string input in a database, and providing at least a portion of the string input to a processing circuit. The computer-implemented method also includes receiving, from the processing circuit, a code associated with the string input, causing a display in the client device to display the code for the user and providing, to the remote client, a result associated with the technical input data.

In a second embodiment, a computer-implemented method includes receiving, in a server, a request for a service from a remote client, wherein the service comprises an access to a model that processes a technical input data. The computer-implemented method also includes authorizing a user in the remote client to access a resource in the server, the resource including a memory allocation and a processing thread in the server, wherein the memory allocation and the processing thread are called by an instruction in the model. The computer-implemented method also includes providing to the remote client a secure communication channel for handling data transmissions between the server and the remote client and receiving the technical input data from the remote client via the secure communication channel. The computer-implemented method also includes processing the technical input data with the processing thread in the server using the memory allocation, and providing, to the remote client, a result associated with the technical input data.

In a third embodiment, a system includes a memory storing instructions, and one or more processors configured to execute the instructions to cause the system to receive a string input from a client device, the string input being part of a technical chart for a user of the client device. The user is associated with a healthcare provider and the string input comprises a healthcare report of a patient. The one or more processors also execute instructions to standardize the string input according to a set of technical rules, storing the string input in a database, and to provide at least a portion of the string input to a processing circuit. The one or more processors also execute instructions to receive a code associated with the string input, and causing a display in the client device to display the code for the user.

In a fourth embodiment, a non-transitory, computer-readable medium stores instructions which, when executed by a processor in a computer, cause the computer to perform a method. The method includes receiving a string input from a client device, the string input being part of a technical chart for a user of the client device, wherein the user is associated with a healthcare provider and the string input comprises a healthcare report of a patient. The method also includes standardizing the string input according to a set of technical rules, storing the string input in a database, providing at least a portion of the string input to a processing circuit, receiving, from the processing circuit, a code associated with the string input, and causing a display in the client device to display the code for the user.

In yet another embodiment, a system includes a means for storing instructions, and a means to execute the instructions to cause the system to receive a string input from a client device, the string input being part of a technical chart for a user of the client device. The user is associated with a healthcare provider and the string input comprises a healthcare report of a patient. The means to execute instructions also executes instructions to standardize the string input according to a set of technical rules, storing the string input in a database, and to provide at least a portion of the string input to a processing circuit. The means to execute instructions also executes instructions to receive a code associated with the string input, and causing a display in the client device to display the code for the user.

### BRIEF DESCRIPTION OF THE DRAWINGS

FIG. 1 illustrates a system architecture with client devices in a clinic communicatively coupled with a server through a network, for augmented medical coding, according to some embodiments.

FIG. 2 illustrates a client device and a server as used in the system architecture of FIG. 1, according to some embodiments.

FIGS. 3A-3B illustrate charts for receiving operating characteristic curves associated with ICD codes and CPT codes, according to some embodiments.

FIG. 4 illustrates receiving operating characteristics curves including data points for automated algorithm results and for human expert results, according to some embodiments.

FIGS. 5A-5B illustrate charts for receiving operating characteristic curves associated with ICD codes and CPT codes, according to some embodiments.

FIGS. 6A-6D illustrate a screen shot of a portal for an application using an engine for augmented medical coding, according to some embodiments.

FIG. 7 illustrates a system architecture illustrating an API layer server and a server hosting a web portal application in a network, according to some embodiments.

FIG. 8 illustrates in more detail the server hosting the web portal application, according to some embodiments.

FIG. 9 illustrates steps in a method for providing augmented medical coding in real-time, according to some embodiments.

FIG. 10 illustrates steps in a method for providing a machine learning model to a remote client, according to some embodiments.

FIG. 11 is a block diagram illustrating an example computer system with which the architecture of FIG. 1 can be implemented, according to some embodiments.

In the figures, like reference numerals are associated with like features and steps, unless stated otherwise.

### DETAILED DESCRIPTION

In the United States, about 20% of healthcare expenditures cover processing insurance reimbursements every year. This includes medical billing and insurance related administration activity, which typically involves reimbursing medical businesses for complicated services provided. Accordingly, it is highly desirable for medical insurers to manage payments of thousands (or even hundreds of thousands) of medical bills efficiently. Despite the high cost to process reimbursements, a significant number of errors are made on a regular basis, under current technology. This creates a domino effect of problems for both insurers and medical providers.

Medical coding is a process that a healthcare provider (e.g., a hospital, medical group, solo practitioner, laboratory, and the like) must go through to be reimbursed by any insurer. More generally, the users of an augmented medical coding platform as disclosed herein may also include medical coders, auditors, and other administrators associated with the medical and healthcare industry. An augmented medical coding platform as disclosed herein automatically adjusts or suggests codes as a user enters the data. In addition, an augmented medical coding platform as disclosed herein interactively provides suggestions or edits as the user modifies an entry, essentially working as an extension of the user's own capabilities and level of understanding of technical language. Accordingly, in broad terms, a user of systems and platforms as disclosed herein may or may not have technical medical knowledge. Medical coding is the translation of complex medical evaluations and reports (e.g., doctor's notes) into diagnosis and procedure codes. Medical coding is the vehicle by which medical providers can properly communicate their work to payers (e.g., the insurance companies providing reimbursement). Current approaches result in medical coding error rates of about 30%. Insurers, medical providers, and patients all suffer from these errors, as follows.

For insurers, errors create fraudulent payments, overpayments, erroneous capitation modeling, increased need to run audits, and erroneous population health statistics. For example, the Department of Health and Human Services has reported a 44% error rate in E/M level codes alone, resulting in an endless stream of audits and lawsuits.

For medical providers, errors may create at least one of three problems: 1) delayed revenue due to mismatched coding, 2) lost revenue due to down-coding, and 3) legal risk due to up-coding. With the profit margin of many health systems in the 5% range, errors at this level can put these establishments dangerously close to bankruptcy, leaving patients in those communities with nowhere to go for treatment.

For patients, incorrect coding often results in unanticipated and unexpected bills, delayed authorizations for treatment, and erroneous or unnecessary treatment. In some cases, patients are at the mercy of an initial codification and documentation in their provider's notes for proper future care. When the initial codification and documentation is incorrect, patients may have a steep curve to overcome the costly consequences.

Today, medical coding is an overwhelmingly manual process with limited automation. Usually a medical establishment will hire a medical coder (e.g., a person) to manually code medical evaluations. Medical coders may be trained at trade schools, but can also be self-taught. They can be certified through the American Academy of Professional Coders (AAPC). However, certification is not required to legally perform the task. Medical coders are trained to read and understand dense clinical documentation and then distill the diagnosis of a patient into a 7-digit alphanumeric “ICD-10” diagnosis code. They are also trained to distill the service delivered to the patient into a 5-digit alphanumeric “CPT” code. There are about 70,000 ICD codes to pick from, and about 70,000 CPT codes to pick from, according to the Centers for Disease Control. Combined, these codes are used to bill the insurance company for reimbursement. These codes are also used by payers and governmental organizations to monitor population health, detect fraud, and to do actuarial modeling and other statistical modeling. In some cases, doctors often perform the coding themselves, at least partially, despite receiving no training on medical coding. In some instances, an electronic health records system in a clinic may include a search bar tool where a healthcare provider (e.g., doctor, nurse, and the like) can search for and select seemingly appropriate codes from a drop-down list. In some instances, there may be dozens of codes resulting from a search, so the doctor or practitioner scrolls through, reads, and comprehends each entry in the list, and then selects the correct code.

Payers (e.g., insurance companies) often hire auditors to audit the coding of medical providers. However, the hired auditors have the same subjectivity, bias, and human-error problems as the medical practitioners themselves, if not worse. In some embodiments, payers have either coders or other medical personnel read doctors notes and select proper codes to inject into actuarial models. In some embodiments, a payer searches for patterns of miscoding, errors, and omissions in the coding provided by a healthcare practitioner to detect fraud.

Over two-thirds of medical providers in the United States use an electronic health records (EHR) system to digitize and document their clinical activity. This number grows every year. In fact, the Patient Protection and Affordable Care Act (PPACA) aims to ensure that every single practicing provider in the United States uses an EHR system. As of 2015, healthcare practitioners that do not demonstrate the “meaningful use” of an EHR will be penalized financially with a 1% reduction in reimbursement.

Current methods for software-assisted medical coding include selecting from a list in a dropdown menu or search bar of an EHR platform, or using Computer Assisted Coding (CAC) software which provides a series of steps to algorithmically determine/assist with medical coding. However, these platforms lack a semantic understanding of the text input from the practitioner. Therefore, these platforms tend to offer mechanic responses based on a few word matches, rather than a comprehensive grammatical and semantic understanding of the practitioner's input. Accordingly, while current techniques may enormously simplify the work of the practitioner or coder, they suffer the same bias problems as manual coding techniques.

To resolve the above technical problems, embodiments as disclosed herein make use of artificial intelligence technology to expedite the flow of medical disbursements and substantially remove the current error prone techniques. More specifically, embodiments as disclosed herein use natural language processing (NLP) tools to associate medical coding to healthcare reports provided by healthcare personnel and thus more accurately reflect the cost of a medical procedure as well as its medical necessity. Embodiments of the present disclosure offer the added advantage of a verifiable system that renders reproducible results. In some embodiments, an NLP tool includes convolutional neural networks (CNN) and transformer based techniques to solve the shortcomings of software tools in medical coding and billing by providing accurate predictions with artificial intelligence that increase their performance as more data is included in the system. In some embodiments, an NLP tool includes a statistical analysis package.

Embodiments as disclosed herein use artificial intelligence techniques to provide augmented medical coding. Moreover, some embodiments make use of computer networking advances to ensure a real-time feedback to the medical coding process. In addition, some embodiments may provide clinical documentation improvement suggestions based on historical data and medical outcomes stored in a database.

In some embodiments, a side bar integrated widget may be included in a display for an EHR platform where a user is viewing medical records for one or more patients. The side bar widget may display medical codes associated with the patient data per artificial intelligence algorithms capturing information from the EHR platform, as disclosed herein. Further, in some embodiments the sidebar widget may include an option for the user to export the medical codes to the EHR platform, if accepted. Moreover, the sidebar widget may include suggestions to add to the medical record, or even of actions to take to improve a healthcare outcome for the patient.

Embodiments consistent with the present disclosure provide a technical solution to the above technical problems arising in the field of healthcare computer networks by substantially reducing the time cost for a healthcare professional to navigate and fill out documentation in an EHR platform. Embodiments as disclosed herein provide a solution for doctors and other personnel by reducing the number of clicks for picking and selecting medical codes in EHR platforms. This also substantially reduces the number of involuntary human errors produced by tiresome repetitive input in a typical EHR platform. This in turn translates into a benefit because healthcare professionals are able to perform patient care more efficiently. For example, embodiments as disclosed herein may reduce a five to eight (5-8) click process to obtain a medical code (which typically accrue to at least a few diagnosis codes and at least one procedure code), into a zero to two (0-2) click process per medical code (depending on whether or not a preselecting option for the code is activated on the sidebar widget, by the doctor). In some embodiments, the sidebar widget can preselect codes for the user (e.g., doctor or clinician) depending on the confidence level of the machine learning algorithm being used and the input data. Such scenario may lead to a “no click” experience for the user.

In some scenarios, doctors may use a medical code suggested or selected by the sidebar widget to document a diagnosis, rather than for billing purposes. Accordingly, in some embodiments the sidebar widget suggests codes to the clinician, and with one click, a diagnosis may be added to the legal documentation of the patient encounter. Further, in some embodiments, a medical bill may be updated when the code is posted in the billing section of the EHR platform.

In addition to machine learning and NLP algorithms, some embodiments may implement non-machine learning based and non-natural language based techniques, such as statistical models, or look up tables. For example, in some embodiments a sidebar widget may display a list of frequently used medical codes for the clinician.

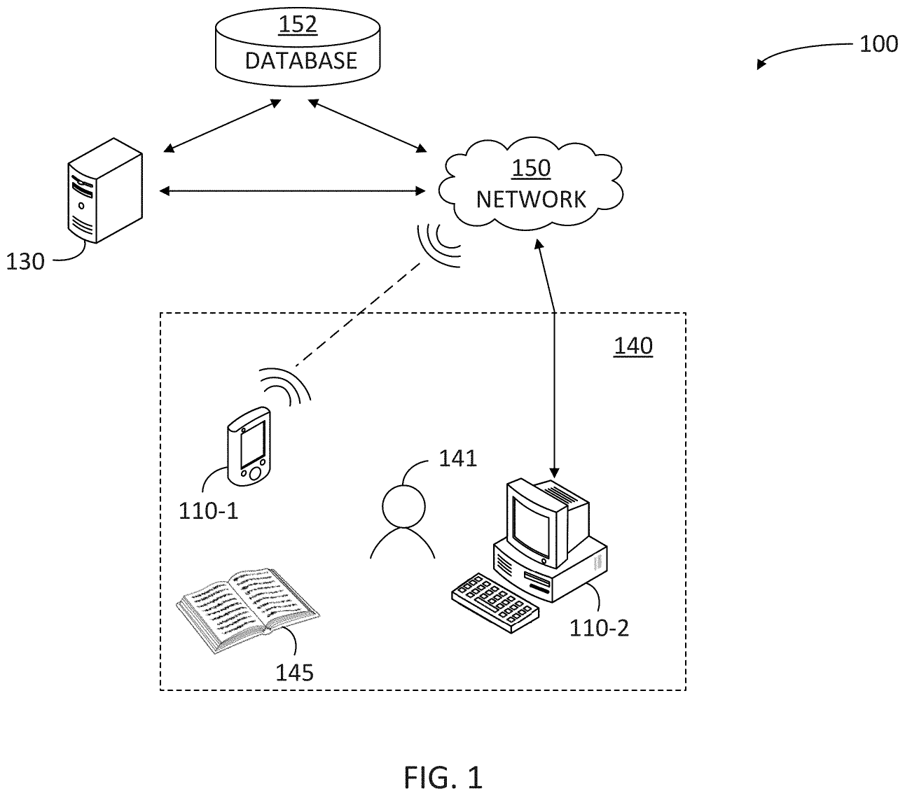

FIG. 1 illustrates a system architecture 100 with client devices 110-1 and 110-2 (hereinafter, collectively referred to as “client devices 110”) in a clinic 140 communicatively coupled with a server 130 through a network 150, for augmented medical coding according to some embodiments. Client devices 110 may include a mobile device 110-1 and/or a desktop computer 110-2. A user 141 (e.g., a healthcare personnel such as a doctor or nurse in a clinic, a medical coder, an administrator, or any other professional associated with a healthcare provider) may prepare a healthcare report for a patient, uploading notes from a notebook 145 or responses from a patient from a questionnaire onto an application hosted by server 130 and running in any one of client devices 110. A database 152 in system architecture 100 may be accessed by server 130 directly, or via network 150. Database 152 may include historical data collected from multiple patients across multiple clinics 140 over an extended period of time.

Providers using an EHR will typically dictate, type, or upload a pre-typed version of their patient evaluations. Given the way that EHRs are designed: as a repository of data that is collected from the point of patient care to the point of billing for reimbursement, providers are often burdened with spending too much time with data entry tasks. The data entered must be placed into clearly labeled fields to (a) ensure the provider has not forgotten to document a critical piece of information, and (b) so that personnel downstream from the provider can utilize the information efficiently. Unfortunately, this has created a situation where today, medical providers are spending over 50% of their time in EHR data entry.

Exhaustive clinical documentation drives medical coding. A medical coder may not codify a diagnosis or procedure code that is not supported by the clinical documentation. Moreover, improper clinical documentation can be the basis for audits that can result in severe penalties for medical providers. Proper clinical documentation is also important for the quality of patient care delivered. Doctors, nurses, administrators, payers, and other entities all depend on the clinical documentation posted to make decisions on the patient's care. Incomplete or erroneous documentation can place a patient's life at risk.

Given the nature of EHRs, the importance of clinical documentation, and the challenges at hand, artificial intelligence will play an increasingly critical role in reducing the time that busy providers spend on data entry while simultaneously enhancing the quality of clinical documentation produced by those providers.

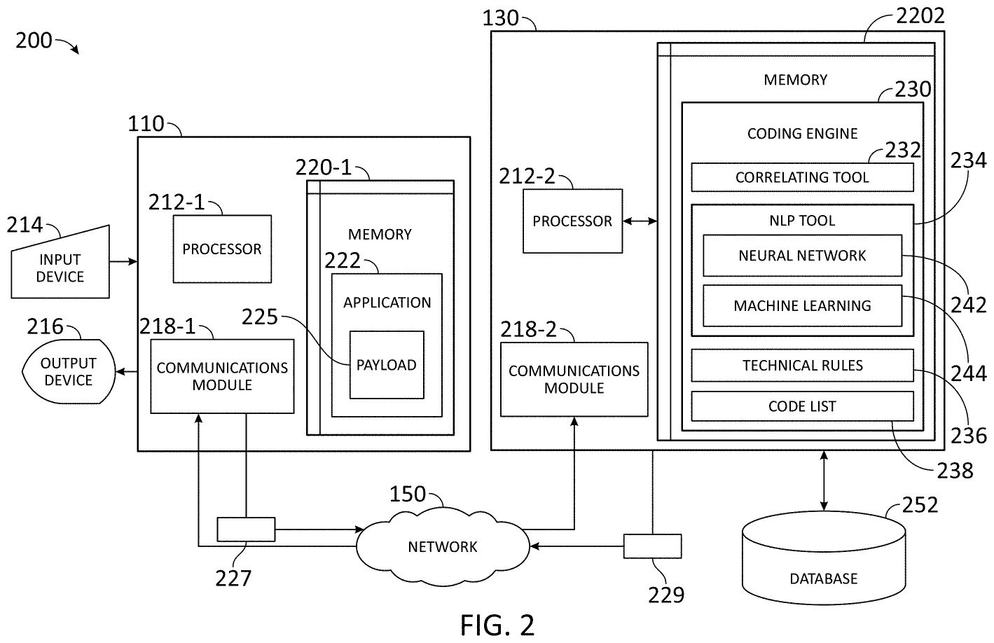

FIG. 2 illustrates architecture 200 including a client device 110 and a server as used in a system architecture as disclosed herein (e.g., system architecture 100), according to some embodiments. Client device 110 and server 130 are communicatively coupled over a network 150 via respective communications modules 218-1 and 218-2 (hereinafter, collectively referred to as “communication modules 218”). Communications modules 218 are configured to interface with network 150 to send and receive information, such as data, requests, responses, and commands to other devices on network 150. In some embodiments, communications modules 218 may include, for example, modems or Ethernet cards. Client device 110 may be coupled with an input device 214 and with an output device 216. Input device 214 may include a keyboard, a mouse, a pointer, or even a touch-screen display that a user (e.g., a consumer, a medical provider, a medical practitioner, a medical coder, an administrator, or any other professional associated with a healthcare provider) may utilize to interact with client device 110. Likewise, output device 216 may include a display and a speaker with which the user may retrieve results from client device 110. Client device 110 may also include a processor circuit 212-1, configured to execute instructions stored in a memory circuit 220-1, and to cause client device 110 to perform at least some of the steps in methods consistent with the present disclosure. Memory circuit 220-1 may further include an application 222 including specific instructions which, when executed by processor circuit 212-1, cause a payload 225 hosted by server 130 to be displayed for the user. Accordingly, application 222 may be installed by server 130 and perform scripts and other routines provided by one or more servers 130 coupled to network 150. More generally, Application 222 may be a browser extension, a mobile application, or a desktop application. In some embodiments, application 222 may be configured to display a payload 225 provided by a coding engine 230. Payload 225 may include a code 229 for a medical condition, symptom, or another healthcare related data, presented to the user of client device 110 by server 130. In some embodiments, the user may select at least some of the codes 229, or provide feedback 227 to server 130 by activating buttons and other fields in payload 225, stored in memory circuit 220-1.

Server 130 includes a memory circuit 220-2, a processor circuit 212-2, and communications module 218-2. Processor circuit 212-2 is configured to execute instructions, such as instructions physically coded into processor circuit 212-2, instructions received from software in memory circuit 220-2, or a combination of both. Memory circuit 220-2 includes coding engine 230 for providing, to payload 225, at least one or more codes associated with a healthcare data input (e.g., feedback 227) by the user through application 222. Coding engine 230 may also provide clinical improvement suggestions to the user of the client device in the payload. Hereinafter, processor circuits 212-1 and 212-2 will be collectively referred to as “processor circuits 212,” and memory circuits 220-1 and 220-2 will be collectively referred to as “memory circuits 220.”

In some embodiments, coding engine 230 integrates payload 225 using a correlating tool 232 and an NLP tool 234. NLP tool 234 may include a nonlinear algorithm such as a neural network 242 or a machine learning algorithm 244. In some embodiments, NLP tool 234 is configured to organize and structure knowledge to perform tasks such as automatic summarization, translation, named entity recognition, relationship extraction, speech recognition, and topic segmentation. Memory 220-2 may further complement coding engine 230 with a set of technical rules 236 (e.g., medical procedures, physiological information, drug prescriptions and databases, therapeutic libraries, and the like). Memory 220-2 may also include a code list 238, provided by a technical panel, or a convention of a professional practice.

In some embodiments, neural network 242 may include a convolutional neural network (CNN). A CNN architecture may include filters of size one through five on the text data with multiple (e.g., up to 512 or more) filters activated by ReLU, followed by a max pooling layer for each filter size (other applications may work with fewer filter or more filters, depending on the application). A structured data, or a categorical and numerical data, is provided through a dense layer of size 128 activated by a ReLU function with a 0.8 dropout. It will be apparent that a dense layer of different size and a lower or higher dropout rate may be used to obtain better results in different applications, according to some aspects of the present disclosure. Some embodiments include an aggressive dropout to avoid overfitting to categorical information due to the number of features relative to the number of observations. In addition, if available, some embodiments include use labels from prior encounters as a feature. Some embodiments concatenate multiple max pooling layers (one for each filter size), the categorical information, and the prior labels, and feed it through two dense layers followed by 0.5 dropout. In some embodiments, the model is trained for a maximum of 100 epochs. The training stops early when the validation loss has minimized and does not improve for 30 epochs subsequent to this minimum. The number of epochs may also be smaller or higher, depending on the application and the computational capabilities available to the designer. In some embodiments, this architecture includes text-only inputs, categorical-only inputs, and prior labels only as part of our ablation studies. The above parameters and values are exemplary of one embodiment only, and it should be understood that a range of values may be selected for different applications consistent with the present disclosure.

In some embodiments, NLP tool 234 may include a bidirectional encoder representation (BERT) for text classification tasks. Accordingly, some embodiments include an exercise of transfer learning by replacing the sequence classification component of a CNN model above with BERT. In some embodiments, uncased BERT base pre-trained weights are used to train a multi-label text classification model on a text data for 100 epochs. Some embodiments include BioBERT weights, which retrain the BERT weights on a corpus of biomedical texts (namely PubMed abstracts). In some embodiments, at least some of the information in memory 220-2 (e.g., technical rules 236 or code list 238) may be stored in a database 252 that may be external to and communicatively coupled with (via network 150) server 130.

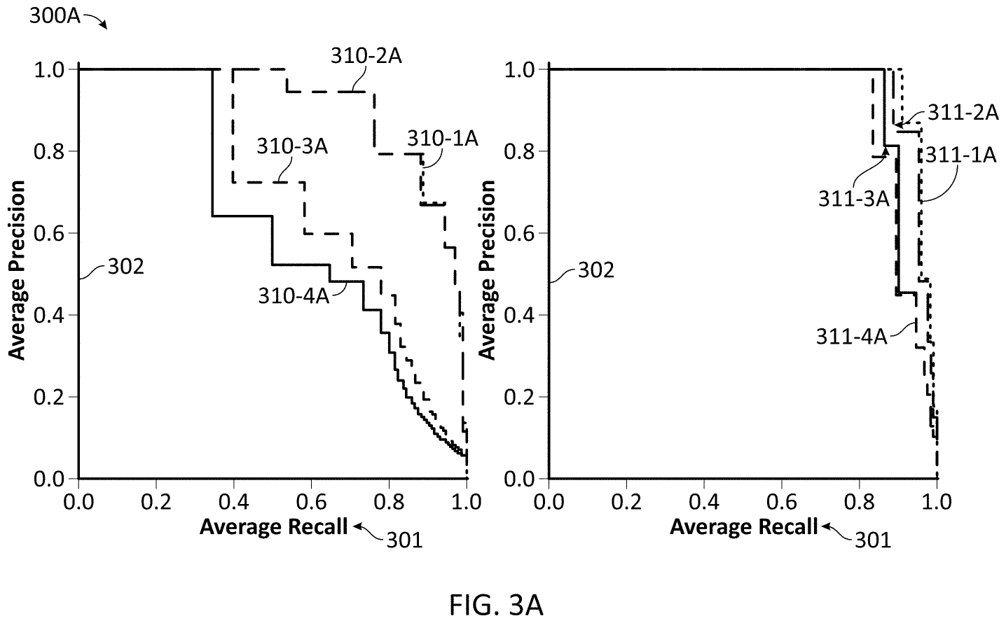

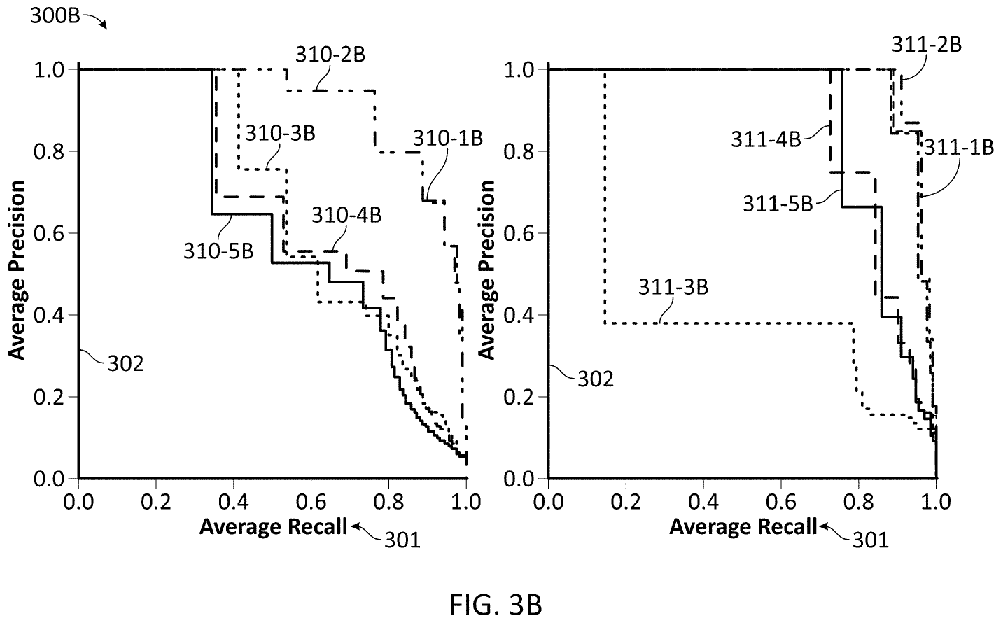

FIGS. 3A-3B illustrate charts 300A and 300B (hereinafter, collectively referred to as “charts 300”) for receiving operating characteristic (ROC) curves associated with ICD codes and CPT codes, according to some embodiments. In charts 300, the abscissae 301 (X-axis) corresponds to the average “recall” (e.g., true code assessment rate) and the ordinate 302 (Y-axis) corresponds to an average precision (e.g., how distinct was the code assignment from any other code, or how close was the code assignment to the “ideal” code assignment value). A quality factor for ROC curves in charts 300 may be defined as the “area under the curve” (AUC).

To calculate AUC of Average Precision/Average Recall (AUCAPAR), the number of labels (k) predicted can always be selected by the modeler, which opens the possibility for “cherry-picking” a k that shows the best performance. In some embodiments, a coding engine computes average precision and average recall at all values of k and calculates an AUC of the resulting precision/recall curve. This summarizes how the model performs across all k. In some embodiments, the coding engine defines average precision (AP(k)) and average recall at k (AR(k)) as follows:

$$\begin{matrix}AP ( K ) = \frac{1}{N} {\sum }_{i = 1}^{N} {P}_{i} ( k ) & ( 1 ) \\ AR ( K ) = \frac{1}{N} {\sum }_{i = 1}^{N} Δ {R}_{i} ( k ) & ( 2 )\end{matrix}$$

where Pi (k), Ri (k), and N are defined as above. It can be shown that average recall will mechanically increase to 1 as k→K, and therefore by its own is not a reliable metric. However, AP(k) and AR(k) vary with a common k, so we can therefore calculate an AUC of the resulting relationship:

AUC_{APAR}=∫₀¹AP(AR)·dAR (3)

This metric summarizes the performance of a model across all values of k, avoiding potential pitfalls that come from attempting to select a k to summarize the predictive power, and is based on the well-known AP(k) and AR(k) metrics for multi-label classification.

Chart 300A includes ROC curves 310-1A, 310-2A, 310-3A, and 310-4A (hereinafter, collectively referred to as “ROC curves 310A”), and ROC curves 311-1A, 311-2A, 311-3A, and 311-4A (hereinafter, collectively referred to as “ROC curves 311A”) for a convolutional neural network model of an international classification of diseases and current procedural terminology codes having different area under the curve, according to some embodiments. ROC curves 310A are associated with ICD codes, and ROC curves 311A are associated with CPT codes. Each of ROC curves 310A and 311A is associated with a different technique or model to obtain the associated code: concatenation (ROC curves 310-1A and 311-1A, with AUC 0.916 and 0.973, respectively), text (ROC curves 310-2A and 311-2A, with AUC 0.915 and 0.966, respectively), prior labels (ROC curves 310-3A and 311-3A with AUC 0.697 and 0.932, respectively), and categorical (ROC curves 310-4A and 311-4A, with AUC 0.623 and 0.932, respectively).

Chart 300B includes ROC curves 310-1B, 310-2B, 310-3B, 310-4B, and 310-5B (hereinafter, collectively referred to as “ROC curves 310B”), and ROC curves 311-1B, 311-2B, 311-3B, 311-4B, and 311-5B (hereinafter, collectively referred to as “ROC curves 311B”) for a convolutional neural network model of an international classification of diseases and current procedural terminology codes having different area under the curve, according to some embodiments. ROC curves 310B are associated with ICD codes, and ROC curves 311B are associated with CPT codes. Each of ROC curves 310B and 311B is associated with a different technique or model to obtain the associated code: BioBERT (ROC curves 310-1B and 311-1B, with AUC 0.918 and 0.963, respectively), CNN (ROC curves 310-2B and 311-2B, with AUC 0.916 and 0.973, respectively), keywords (ROC curves 310-3B and 311-3B, with AUC 0.666 and 0.422, respectively), NBayes (ROC curves 310-4B and 311-4B with AUC 0.663 and 0.866, respectively), most frequent (ROC curves 310-5B and 311-5B, with AUC 0.620 and 0.865, respectively).

Charts 300 may be obtained by a coding engine including a correlating tool and an NLP tool in combination with technical rules and a code list, as disclosed herein (cf coding engine 230, correlating tool 232, NLP tool 234, technical rules 236, and code list 238). In some embodiments, the NLP tool may include algorithms such as neural network algorithms and machine learning algorithms (cf. neural network 242 and machine learning 244). In some embodiments, the NLP tool includes an (AI) Predictive Modeling that takes in as input a medical chart (both structured and unstructured data) and outputs predictions including, but not limited to: diagnosis codes (ICD-10), procedure codes (CPT), evaluation/management (E/M) codes, and clinical documentation improvement (CDI). The mean average precision (mAP) and capabilities of this model are illustrated in Table 1, below.

TABLE 1
Model Mean Average Precision (mAP)

| | Clinic | | Expert | |
|---|---|---|---|---|
| Model: | ICD | CPT | ICD | CPT |
| Most Frequent | 0.656 | 0.756 | 0.427 | 0.157 |
| Keywords | 0.704 | 0.499 | 0.513 | 0.107 |
| NBayes | 0.707 | 0.848 | 0.456 | 0.177 |
| CNN | 0.944 | 0.880 | 0.723 | 0.306 |
| BERT | 0.947 | 0.863 | 0.711 | 0.291 |
| BioBERT | 0.946 | 0.859 | 0.714 | 0.283 |

The coding engine may formulate mAP as follows:

$$mAP ( K ) = \frac{1}{N} {\sum }_{i = 1}^{N} {\sum }_{k = 1}^{K} {P}_{i} ( k ) \cdot Δ {R}_{i} ( k ) \qquad ( 4 )$$

Where K is the total number of labels, N is the number of instances, and Pi (k) is the precision up to label k for instance, i. ΔRi (k) is the change in the recall between label k and k−1 for instance i. In essence, mAP averages the precision values across all k as long as increasing k increases the average recall as well.

The mAP values are calculated at the maximum number of labels possible. For ICD, this is 46 labels, and for CPT, this is 12. Clinic columns calculate mAP where the ground truth assumption is the clinic's data. The “Expert” columns assume that that the ground truth is that of the expert medical coders. To assess the bias (e.g., “subjectivity”) of the models, a weighted score F₁ may be associated with each coder, as illustrated in Table 2, below.

There are several noteworthy points here. As measured by both AUCAPAR and mAP, our deep learning models using CNN, BERT, and BioBERT outperform all other predictive approaches. In addition, BERT approaches may be more desirable for ICD predictions, while CNN may be more desirable for CPT predictions. In general, there is minor variability in performance between our deep learning models, and there is a clear performance distinction between the group of non-deep learning approaches and deep learning ones. As discussed above, however, these are only comparisons to our holdout sample.

TABLE 2
Weighted F₁ Scores for Subjectivity Studies

| | Coder | F₁^{Codes} | F₁^{NoCodes} | Δ | S_{Δ} | p-value |
|---|---|---|---|---|---|---|
| ICD codes revealed: | | | | | | |
| Before Coding | 1 | 0.708 | 0.798 | −0.089 | 5.7e−5 | <1e−20 |
| | 2 | 0.559 | 0.698 | −0.139 | 5.8e−5 | <1e−20 |
| After Coding | 1 | 0.799 | 0.812 | −0.013 | 1.4e−5 | <1e−20 |
| | 2 | 0.697 | 0.692 | 0.005 | 5e−6 | <1e−20 |
| CPT codes revealed: | | | | | | |
| Before Coding | 1 | 0.710 | 0.796 | −0.086 | 1.8e−3 | <1e−20 |
| | 2 | 0.558 | 0.700 | −0.143 | 1.8e−3 | <1e−20 |
| After Coding | 1 | 0.799 | 0.813 | −0.014 | 4.2e−4 | <1e−20 |
| | 2 | 0.698 | 0.693 | 0.005 | 1.5e−4 | <1e−20 |

Table 2 shows how each coder's weighted F₁ scores changed under two subjectivity studies. The first experiment revealed the ICD and CPT codes that the clinic had assigned to an encounter before coders began coding for a sample of 50 charts. The second experiment revealed the codes after the coder had finished coding, and they were asked to revise their codes in light of this information if deemed necessary. F^{Codes}₁ denotes the weighted F₁ score for the coder's labels when they have access to the codes, and F^{NoCodes}₁ without access. Standard errors were calculated with a bootstrap method for 1,000 bootstrap samples. Table 2 indicates results obtained using a CNN model.

TABLE 3

| | ICD | | CPT | |
|---|---|---|---|---|
| Labels: | Clinic | Expert | Clinic | Expert |
| Predictor: | | | | |
| Coder 1 | 0.66 | 0.55 | 0.37 | 0.47 |
| Coder 2 | 0.66 | 0.53 | 0.43 | 0.57 |
| Coder 3 | 0.68 | 0.57 | 0.33 | 0.44 |
| Expert | 0.50 | — | 0.35 | — |
| Clinic | — | 0.57 | — | 0.52 |
| Model | 0.89 | 0.60 | 0.70 | 0.54 |

Table 3 shows the average F₁ scores of coders and CNN model against the clinic's (e.g., data's) and expert's labels as ground truth. This table shows how human coders' labels fared against concatenated CNN model for a 500 instance subset of the test data. Coders were not provided the labels in the data and coded independently of other coders. Values in bold indicate the best predictor for the labels in question. Tables 1, 2, and 3 illustrate that through the use of unsupervised learning and self-attention architectures, the coding engine processes large-scale, unlabeled clinical data and extracts meaningful semantic content, providing a learned representation that is generalizable and useful for downstream classification tasks. In some embodiments, this is a pre-trained model. For a given client, the pre-trained model is then fine-tuned/trained on the client's specific historical data (clinical notes, categorical information, and billing history). At this stage, the model is able to provide accurate predictions that can be integrated as a side-bar app, auditing report, or in an API layer. Over time, the model keeps track of the user's selections and interactions to continually learn through active learning techniques to better improve model predictions. The specific numbers presented in Tables 1, 2, and 3 are exemplary only and not limiting. Accordingly, any range of values for each of the listed parameters may be selected for other applications in view of the specific conditions and nature of a problem, and the computational capabilities of the users.

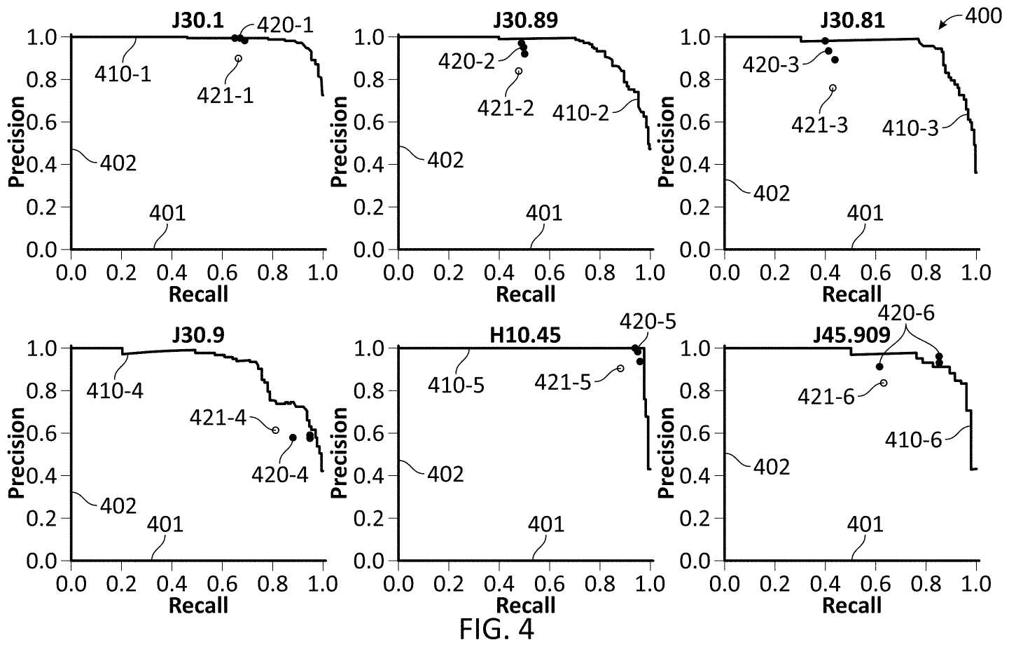

FIG. 4 illustrates charts 400 with ROC curves 410-1, 410-2, 410-3, 410-4, 410-5, and 410-6 (hereinafter, collectively referred to as “ROC curves 410”) including data points 420-1, 420-2, 420-3, 420-4, 420-5, and 420-6 (collectively referred to, hereinafter, as “data points 420”) for automated algorithm results 421-1, 421-2, 421-3, 421-4, 421-5, and 421-6 (collectively referred to, hereinafter, as “data points 421”) for human expert results, according to some embodiments. In charts 400, the abscissae 401 (X-axis) corresponds to the average “recall” and the ordinate 402 (Y-axis) corresponds to an average precision. By comparing the location of data points 420 with the location of data points 421, it is clearly seen that the automated algorithm results in embodiments consistent with the present disclosure are systematically superior to the human expert results.

Each of ROC curves 410 is associated with one code that may be a high frequency code in healthcare practice. For example, and without limitation: ROC curve 410-1 may be associated with code J30.1, ROC curve 410-2 may be associated with code J30.89, ROC curve 410-3 may be associated with code J30.81, ROC curve 410-4 may be associated with code J30.9, ROC curve 410-5 may be associated with code H10.45, and ROC curve 410-6 may be associated with code J45.909.

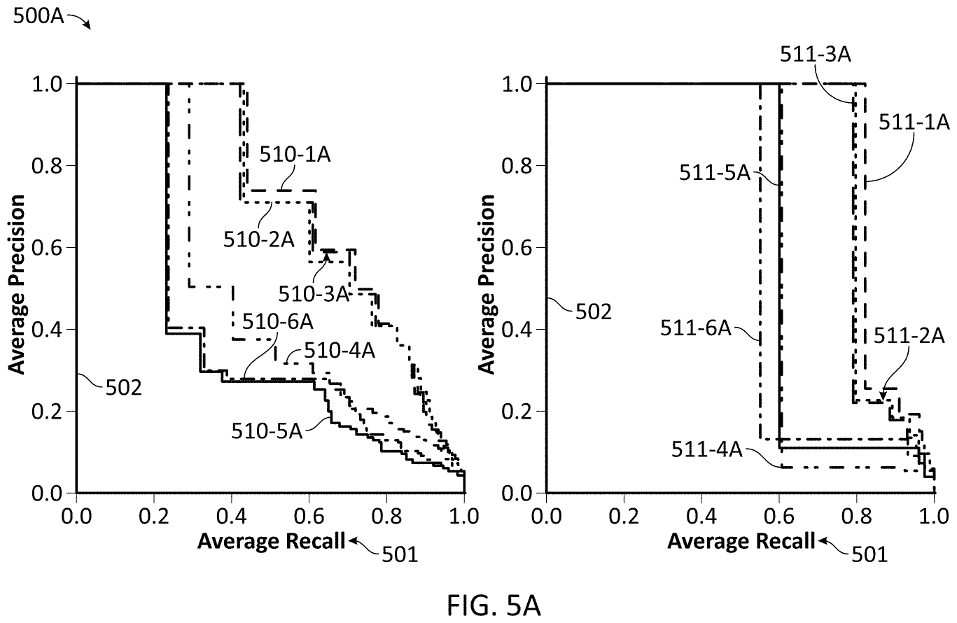

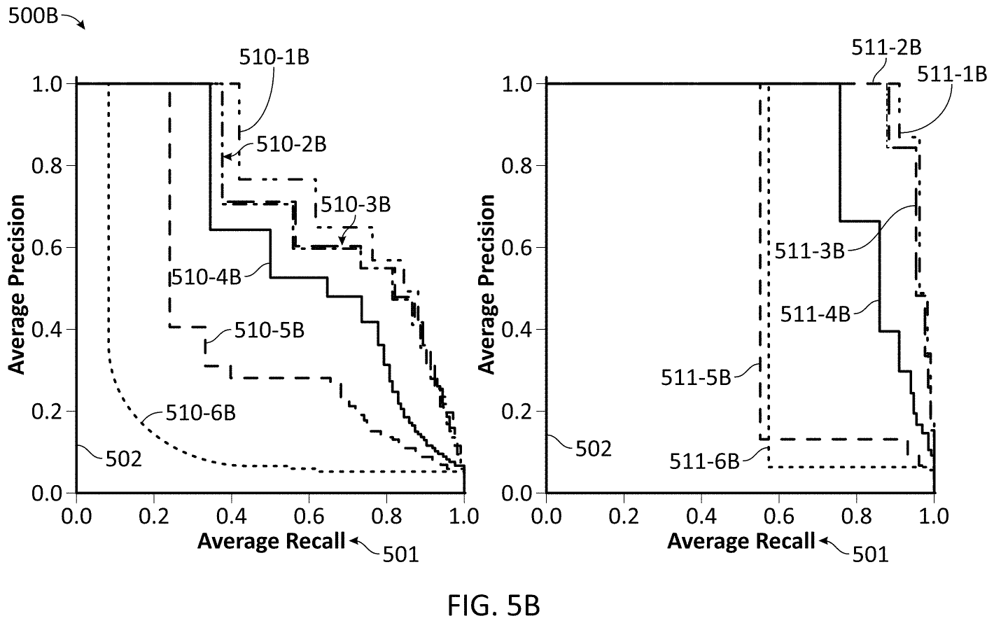

FIGS. 5A-5B illustrate charts 500A and 500B (hereinafter, collectively referred to as “charts 500”) for receiving operating characteristic (ROC) curves associated with ICD codes and CPT codes, according to some embodiments. In charts 500, the abscissae 501 (X-axis) corresponds to the average “recall” and the ordinate 502 (Y-axis) corresponds to an average precision.

Chart 500A includes ROC curves 510-1A, 510-2A, 510-3A, 510-4A, 510-5A, and 510-6A (hereinafter, collectively referred to as “ROC curves 510A”), and ROC curves 511-1A, 511-2A, 511-3A, 511-4A, 511-5A, and 511-6A (hereinafter, collectively referred to as “ROC curves 511A”) for a convolutional neural network model of an international classification of diseases and current procedural terminology codes having different area under the curve, according to some embodiments. ROC curves 510A are associated with ICD codes, and ROC curves 511A are associated with CPT codes. Each of ROC curves 510A and 511A is associated with a different technique or model to obtain the associated code: CNN (ROC curves 510-1A and 511-1A, with AUC 0.720 and 0.860, respectively), BERT (ROC curves 510-2A and 511-2A, with AUC 0.705 and 0.838, respectively), BioBERT (ROC curves 510-3A and 511-3A, with AUC 0.707 and 0.832, respectively), keywords (ROC curves 510-4A and 511-4A, with AUC 0.495 and 0.635, respectively), most frequent (ROC curves 510-5A and 511-5A, with AUC 0.404 and 0.604, respectively), NBayes (ROC curves 510-6A and 511-6A with AUC 0.419 and 0.604, respectively).

Chart 500B includes ROC curves 510-1B, 510-2B, 510-3B, 510-4B, 510-5B, and 510-6B (hereinafter, collectively referred to as “ROC curves 510B”), and ROC curves 511-1B, 511-2B, 511-3B, 511-4B, 511-5B, and 511-6B (hereinafter, collectively referred to as “ROC curves 511B”) for a convolutional neural network model of an international classification of diseases and current procedural terminology codes having different area under the curve, according to some embodiments. ROC curves 510B are associated with ICD codes, and ROC curves 511B are associated with CPT codes. Each of ROC curves 510B and 511B is associated with a different technique or model to obtain the associated code: CNN (ROC curves 510-1B and 511-1B, with AUC 0.762 and 0.972, respectively), BERT (ROC curves 510-2B and 511-2B, with AUC 0.717 and 0.963, respectively), BioBERT (ROC curves 510-3B and 511-3B, with AUC 0.713 and 0.963, respectively), keywords (ROC curves 510-4B and 511-4B, with AUC 0.143 and 0.605, respectively), most frequent (ROC curves 510-5B and 511-5B, with AUC 0.620 and 0.865, respectively), NBayes (ROC curves 510-6B and 511-6B with AUC 0.419 and 0.604, respectively).

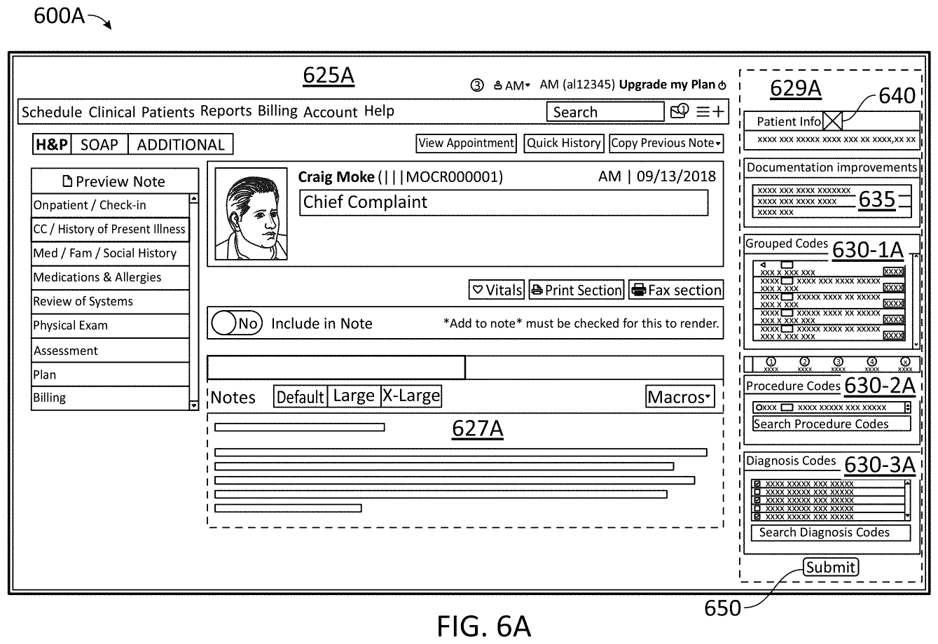

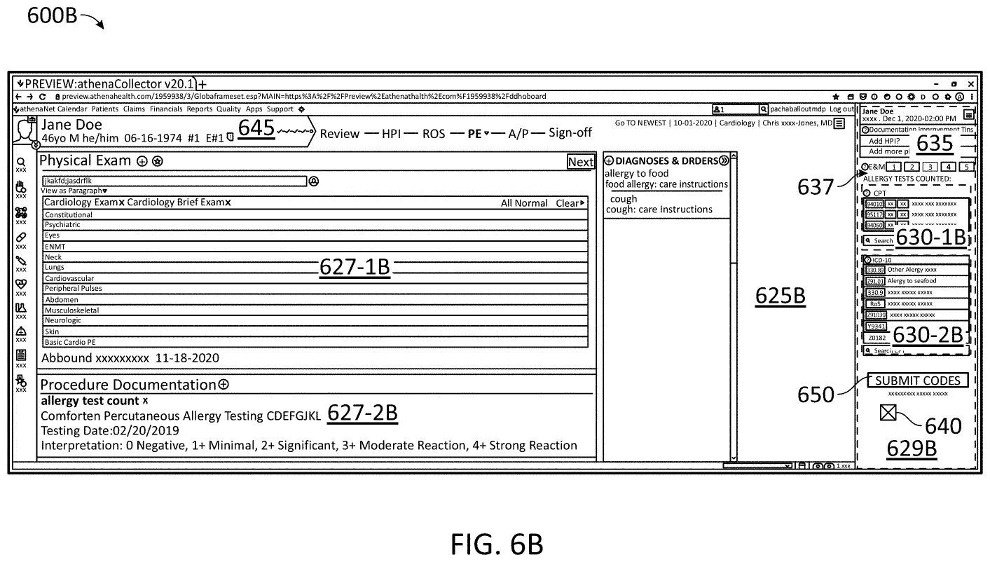

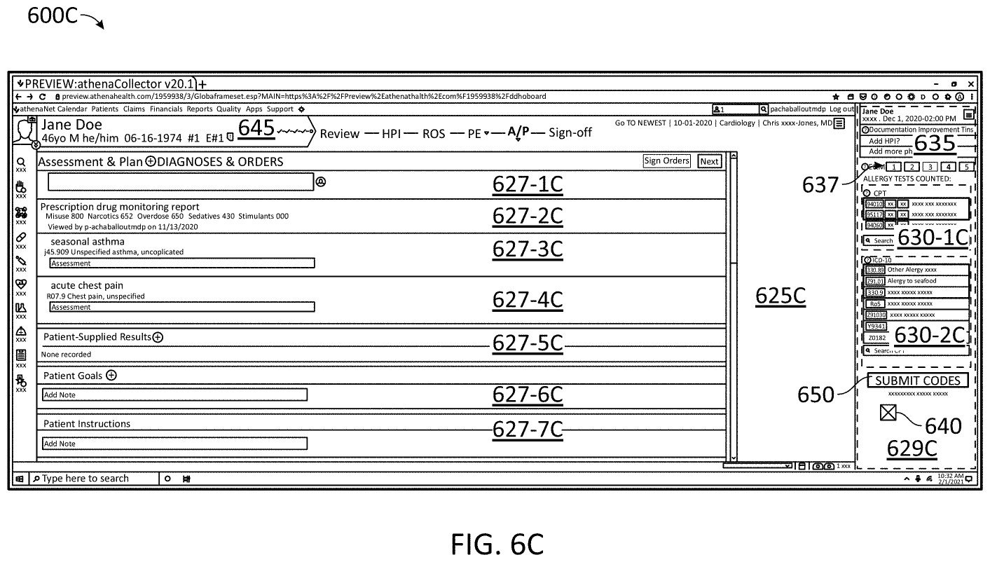

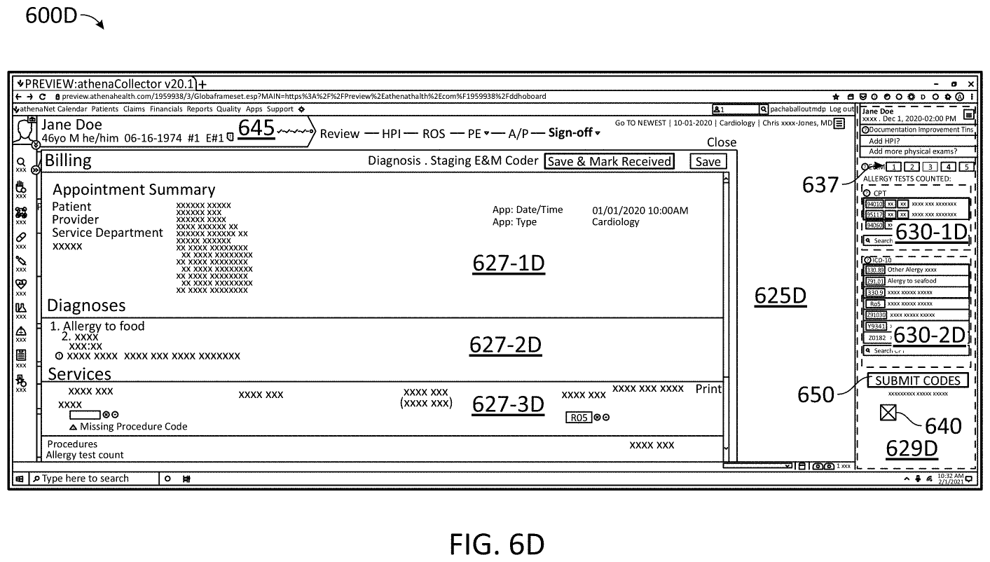

FIGS. 6A-6D illustrate screen shots 600A, 600B, 600C, and 600D (hereinafter, collectively referred to as “screenshots 600”) of electronic health record (EHR) portals 625A, 625B, 625C, and 625C (hereinafter, collectively referred to as “EHR portals 625”) for an application using a coding engine for augmented medical coding, according to some embodiments. In the portal, a text input field 627 enables the user to enter the medical record for a patient. Sidebar application programming interfaces (APIs) 629A, 629B, 629C, and 629D (hereinafter, collectively referred to as “API's 629”), adjacent to text input field 627 provides the user a payload with suggested codes for the condition of the patient according to the text input. In some embodiments, sidebar APIs 629 provide suggested codes (e.g., grouped codes 630-1A, procedure codes 630-2A, and diagnosis codes 630-3A, CPT codes 630-1B, 630-1C, and 630-1D, ICD codes 630-2B, 630-2C, and 630-2D, hereinafter, collectively referred to as “codes 630”) for the user, in real time, e.g., as the user types a new text input in text input field 627. A “submit” button 650 enables the user to transmit codes 630 to EHR portals 625 (e.g., for recordation, or for preparing invoices and treatment recommendations).

Screen shots 600 illustrate a side-by-side usage of EHR portal 625 and sidebar APIs 629. The user accesses/edits the medical record for a given patient encounter in EHR portals 625. As the user is interacting with EHR portals 625, APIs 629 will continually update and display medical charting and billing related information that is relevant for the user. The user can then select codes 630 for a given patient encounter and have at least one or more of codes 630 posted into EHR portals 625. In some embodiments, APIs 629, may continually pull data from the medical chart the user is currently attending to (text input 627), using a browser extension or a desktop application. In some embodiments, APIs 629 also update a database (e.g., database 252) that includes medical chart information with background work processes. In some embodiments, the user may access APIs 629 at two stages: (1) during chart creation and (2) after the chart is completed. During chart creation, the user is continually updating a particular patient encounter in EHR portal 625A. API 629 reads the data input dynamically by the user and passes the data through abstract layers, which eventually get fed to a model in a coding engine (cf. FIG. 2). The model predictions are then displayed in the user interface (UI) of the web application. In some embodiments, the application interface may alert the user to relevant medical coding and billing related information.

A graphic component or payload is displayed as a sidebar to EHR portals 625 in a client device by APIs 629, hosted by an engine for augmented medical coding, according to some embodiments. For a given patient encounter, APIs 629 display accurate predictions from an artificial intelligence (AI) model across ICD-10 diagnosis codes, CPT codes, E/M levels, and clinical documentation improvement (CDI). In some embodiments, E/M codes, while technically service codes (CPT), may be considered separately. For example, in some embodiments, E/M codes may include levels 1 through 5, with 5 being most complex patient evaluation and 1 being least complex patient evaluation. Accordingly, in some embodiments, the higher the level, the higher the reimbursement. In some embodiments, APIs 629 allow a bidirectional communication with EHR portals 625: receiving information (e.g., clinical note or text input field 627) and posting information (codes 630, billing information, and recommendations on documentation improvement 635). Accordingly, documentation improvement 635 may include a suggestion for the healthcare practitioner or user to improve the clinical documentation. Documentation improvement 635 may be presented in the sidebar of API 629 as textual display for assistance to the user to improve of the text input in real-time. In some embodiments, APIs 629 include e/m levels 637 (e.g., 1-5), which may not be the official codes. In some embodiments, API 629 may programmatically convert to the appropriate code which will differ if the patient is new, or a well-established patient. In some embodiments, e/m levels 637 may adjust differences from distinct medical specialties and may be done automatically in the backend.

APIs 629 allow the user to manually search for codes from any screen in EHR portal 625. Some embodiments include a search bar for a CPT procedure code 630-2A, and for ICD-10 codes or diagnosis codes 630-3A. In some embodiments, the search may include machine learning or artificial intelligence algorithms. For example a search bar as provided herein may not include “post partum depression” as an output to the query “depression,” when the patient is not female, or even if the patient is female, the patient may not have been pregnant. Accordingly, a smart search as disclosed herein provides results targeted to the clinic, the doctor, the patient, and an encounter at hand. In some embodiments, the search may include statistical analysis. For example, the system can provide results based on a weigh value for most frequently used codes in the past (by a given user) to appear first in the search result.

In some embodiments, APIs 629 may include a standard mode for illustrating the different healthcare codes, wherein the user selects relevant picks (e.g., CPT/EM and/or one or more diagnosis code, cf. APIs 629-B through D) without a specific grouping or order. In some embodiments, the user may select a “grouped mode” for illustrating healthcare codes (cf. API 629-A). In grouped mode, a user will control the grouping of diagnosis codes to CPT codes, EM codes, and one or more diagnosis codes associated with the CPT/EM code. The user may pick a second CPT/EM code if applicable, and one or more diagnosis codes associated with the second CPT/EM code.

In some embodiments, APIs 629 may include a “follow” feature 640 that allows the sidebar display to “follow” a selected patient for the user (e.g., healthcare practitioner or provider, medical coder, administrator, or any professional associated with a healthcare provider), over multiple patients and over multiple encounters (i.e. dates of service) treated by the user, seamlessly. Accordingly, the user may simply select one or more patients previously interacted with from a pull down menu listing multiple patients that the user is working on in EHR portal 625A. In some embodiments, the payload of API 629 may present a pulldown menu automatically for the user, thus enhancing the interface experience and reducing the time spent by the user on the application. In some embodiments, the user may have logged into a personal EHR portal 625 and toggle patients within a personal account. APIs 629 follow a patient that the user has loaded on EHR 625A, at any given moment.

Screenshot 600A illustrates details of API 629A, including multiple diagnosis coding 630-3A, procedure coding 630-2A, and E/M leveling, the user can click on the predicted codes 630 displayed in API 629A and choose to post these codes into EHR portal 625A. API 629A will then send the selected codes to EHR portal 625A. In some embodiments, API 629A dynamically reads information as it is being added into the medical chart via text input field 627. In some embodiments, after the chart is completed and entered through text input field 627, API 629A displays relevant predictions from the NLP model in the coding engine for a given patient encounter. API 629A thus allows a user to select codes to be sent to EHR portal 625A.

Screenshot 600B illustrates details of API 629B, including CPT codes 630-1B, and ICD codes 630-2B associated with the electronic records of a specific patient 645 selected in EHR portal 625B. EHR portal 625B may include a physical exam field 627-1B, and a procedure documentation field 627-2B. Physical exam field 627-1B may include results from any number of medical exams on the patient, including, but not limited to: constitutional, psychiatric, eyes, ENMT, neck, lungs, cardiovascular, peripheral pulses, abdomen, musculoskeletal, neurologic, skin, and basic cardio PE, among many others. Screenshot 600B illustrates a sample procedure documentation from a specific allergy clinic. Without limitation, this procedure documentation may include any procedure such as: injection, surgery, etc. in other words, this will change from patient to patient, and the same may occur in review of systems, physical exam, and the like. For example, in some embodiments, procedure documentation field 627-2B may include, without limitation, an allergy test count including a testing date, and a listing of results of the test based on a grade scale: 0 negative, 1+ minimal, 2+ significant, 3+ moderate reaction, and 4+ strong reaction.

Screenshot 600C illustrates details of API 629C, including CPT codes 630-1C and ICD codes 630-2C associated with the electronic records of a specific patient 645 selected in EHR portal 625C. EHR portal 625C may include an assessment and plan field 627-1C, a prescription drug monitoring report field 627-2C, a seasonal asthma field 627-3C, an acute chest pain field 627-4C, a patient supplied results field 627-5C, a patient goals field 627-6C, and a patient instructions field 627-7C (hereinafter, collectively referred to as “input fields 627-C”). In some embodiments, one or more of input fields 627-C may be configured to receive a textual input from the user. Accordingly, API 629C may include NLP algorithms that parse the text from input fields 627-C to arrive at CPT codes 630-1C and ICD codes 630-2C (hereinafter, collectively referred to as “codes 630-C”), in addition to documentation improvement tips 635.

Input fields 627-C include diagnoses that a doctor is documenting for a patient evaluation. Without loss of generality, input fields 627-C may include any diagnoses and may change depending on the provider, specialty, patient condition, and the like. In some embodiments, a doctor may use codes 630-C to document the diagnoses. Screenshot 600C documents the diagnoses on input fields 627-C and codes 630-C contain a description of the diagnosis. In some embodiments, codes 630-C, may include code descriptions called “snomed code descriptions.” Accordingly, codes 630-C may be carried over to the billing page, but may not all be used for billing purposes. In some embodiments, patient instructions 627-7C, results 627-5C and goals 627-6C may be unique to the specific EHR for screenshot 600C. Different EHRs may have different input fields 627-C.

Screenshot 600D illustrates details of API 629D, including CPT codes 630-1D and ICD codes 630-2D (hereinafter, collectively referred to as “codes 630-D”) associated with the electronic records of a specific patient 645 selected in EHR portal 625D. EHR portal 625D may include an appointment summary field 627-1D, a diagnoses field 627-2D, and a services field 627-3D. Appointment summary field 627-1D may include metadata such as the name of patient 645, the provider, the service department, and other data related to the insurance plan for patient 645. Diagnoses field 627-2D may include symptoms, diagnostics, and other technical information related to the medical condition of patient 645.

Diagnoses field 627-2D may include a legal documentation of the diagnosis of the patient. Services field 627-3D may include billing “line-items,” which are listed as a group of codes 630-D. In some embodiments, the least common denominator being the procedure codes (which includes e/m and non-e/m procedures). In some embodiments, a dollar amount associated with each procedure code may be included in services field 627-3D. Accordingly, the procedure may be grouped with one or more diagnosis codes. In some embodiments, codes 630-C offer a justification of the procedure or e/m. For example, when a patient is evaluated at consultation e/m level 4 (the procedure), because the patient has cough, fever, and loss of smell, and the diagnosis may include a serious infectious disease (e.g., Covid-19). Not all diagnoses in diagnosis field 627-2D may be used for billing. In some embodiments, a coder or biller may select less than all diagnoses, or other diagnoses different from those illustrated in diagnosis field 627-2D.

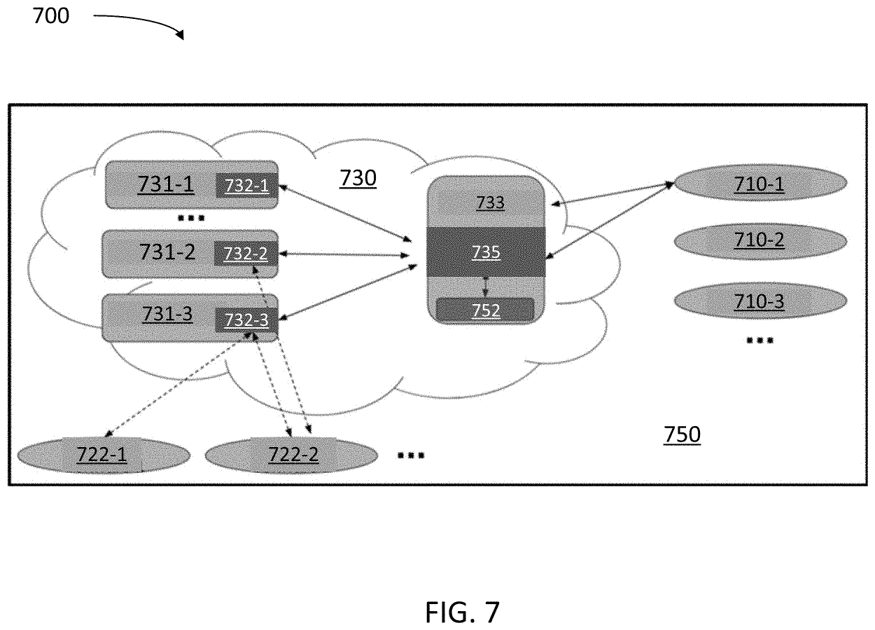

FIG. 7 illustrates a system architecture 700 for a cloud service 730 illustrating API layer servers 731-1, 731-2, 731-3 (hereinafter, collectively referred to as “servers 731”), and a server 733 hosting an application interface 735 in a network 750, according to some embodiments. Servers 731 may each be associated with a different statistical or NLP coding model (ICD, CPT, E/M, CDI, and the like). Coding models for ICD, CPT, E/M, and CDI may include neural network models, artificial intelligence models, machine learning models, statistical models and the like, and may be part of an NLP tool as disclosed herein (cf. FIG. 2). In some embodiments, cloud service 730 may be distributed across multiple processor circuits (e.g., CPUs, GPUs, and the like) over one or more API servers 731. API 735 acts as a front-end to the user and calls APIs 732-1, 732-2, and 732-3 (hereinafter, collectively referred to as APIs 732) of each of the coding models and packages the results on screen, for the user. The components, devices, processing circuits, and memory circuits in the cloud service may be distributed across multiple computer programming unit (CPU) instances and graphic programming units (GPU) instances, located over a broad geographical area. There also is a database 752, which stores both clinical information and model predictions (cf. FIG. 2).

The cloud service may operate in a “software-as-a-service” (SaaS) configuration. In the SaaS configuration, an application interface integrates with multiple EHRs (710-1, 710-2, and 710-3, hereinafter, collectively referred to as EHRs 710) and multiple users on each of EHRs 710. In some embodiments, each one of EHRs 710 may format the same data in different ways. In some aspects, APIs 735 acts as a standardizing interface to translate between EHRs 710 and API servers 731 (e.g., neural network models, artificial intelligence models, machine learning models, and the like), where users can view predictions in a meaningful way. In some embodiments, API 735 converts relevant fields in EHRs 710 to a format that a model in the coding engine can understand through APIs 732. This standardization allows the same models to work on multiple EHRs 710 without having to build a separate model for each individual EHR 710. In addition, cloud service 730 may operate in a “model providing software” configuration, wherein a software provider 722-1 or 722-2 (hereinafter, collectively referred to as “software providers 722”) interested in integrating one or more of the coding models in API servers 731 may interact with the cloud service directly through an API server 731 (e.g., rather than through the application interface or the standardizing interface in server 733). In the “model providing software” configuration, a healthcare practitioner or coder may use a form factor (e.g., APIs 732) available to him/her through a vendor that has authorization to access the models in cloud service 730.

In this configuration, users can send HTTP requests within a familiar JSON or XML format with the relevant medical chart information (numerical, categorical, free-form text, or other modes of data). API 732 will then respond with relevant predictions for the input chart. Accordingly, the user may include a third party software provider 722 that can directly integrate the predictive models from the coding engine in cloud service 730 as disclosed above for their specific applications.

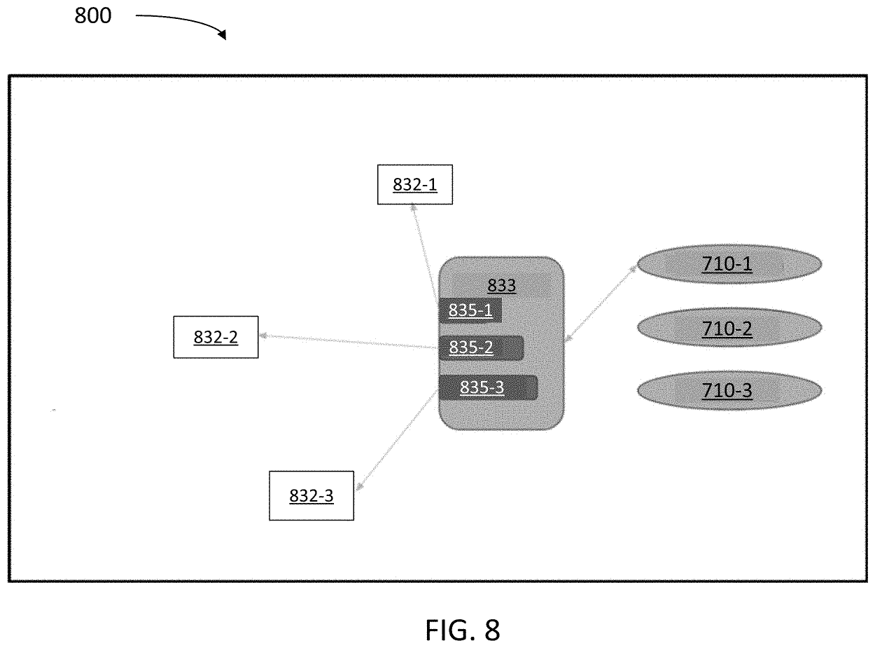

FIG. 8 illustrates in more detail a server 833 hosting standardizing interface, according to some embodiments. In some embodiments, standardizing interfaces 835-1, 835-2, and 835-3 (hereinafter, collectively referred to as “APIs 835”) serves as the universal translator for any clinical (structured or unstructured) information from EHRs 710 to a format understandable by artificial intelligence (AI) Models 832-1, 832-2, and 832-3 (e.g., from API servers 732, hereinafter, collectively referred to as “AI models 832”). Accordingly, APIs 835 handle bidirectional communication from different EHRs 710 and AI Models 832, caching data and information to speed up response times, and handling authentication requests to EHRs 710. Below, some examples of AI models 832 are illustrated without any limitation.

EM model 832-1 interfacing with API 835-1, may include the following script, without limitation of the scope of the present disclosure:

```
{
  “patient_info”: {
    “Visit_type”: “NEW_PATIENT”,
    “dab”: “12-26-1987”
  },
  “Encounter_date”: “5-23-2019”
  “Chief_complaint”: . . .,
  “Assessment”: . . .,
  “ros”: {“eyes”: ..., “ears”: . . .},
  “physical_exam”: {“eyes”: ..., ...,},
  “hpi”: . . .,
  “medical_history”: True,
  “Family_history”: False,
  “social_history”: True
{
```

CDI model 832-2 interfacing with API 835-2, may include the following script, without limitation of the scope of the present disclosure:

```
{ }
  “patient_info”: {
    “Visit_type”: “NEW_PATIENT”,
    “dab”: “12-26-1987”
  },
  “Encounter_date”: “5-23-2019”
  “Chief_complaint”: . . .,
  “Assessment”: . . .,
  “ros”: {“eyes”: ..., “ears”: . . .},
  “physical_exam”: {“eyes”: ..., ...,},
  “hpi”: . . .,
  “Medical_history”: “allergy for ...”,
  “Family_history”: “Father has ...”,
  “social_history”: “Lives in ...,”
}
```

Model 832-3 interfacing with API 835-3, may include the following script, without limitation of the scope of the present disclosure:

```
{
  “patient_info”: {
    “Visit_type”: “NEW_PATIENT”,
    “dab”: “12-26-1987”
  },
  “Encounter_date”: “5-23-2019”
  “Chief_complaint”: category 1
  “Assessment”: . . .,
  “ros”: {“cat1”: ..., “cat2”: . . .},
  “physical_exam”: {“cat1”: ..., ...,},
  “hpi”: . . .,
  “Medical_history”: “allergy for ...”,
  “Family_history”: [cat1, cat2, ...,],
  “social_history”: [cat1, cat2, ...,]
}
```

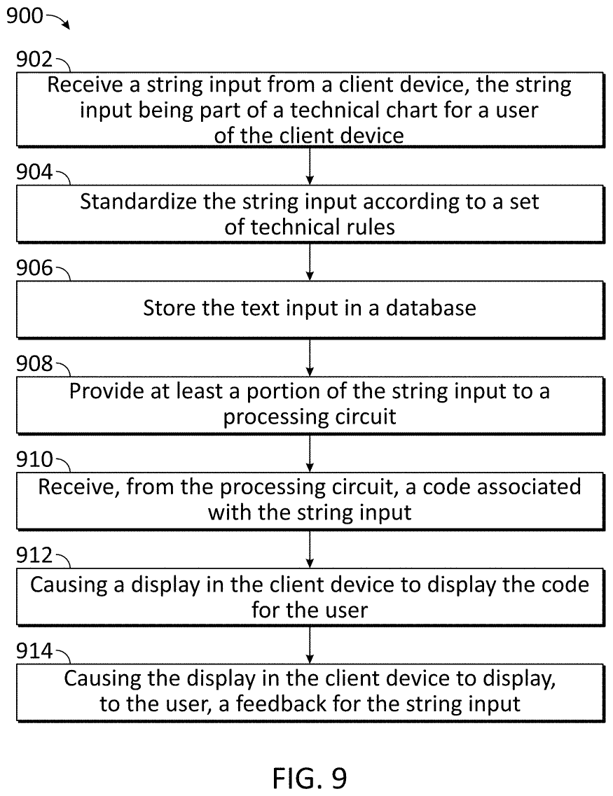

FIG. 9 illustrates steps in a method 900 for providing augmented medical coding in real-time, according to some embodiments. In some embodiments, at least one or more of the steps in method 900 may be performed by one or more devices such as a client device or a server in an architecture as disclosed herein (cf. client devices 110 and servers 130 in system architecture 100). Accordingly, in some embodiments, at least one or more of the steps in method 900 may be performed by an application hosted by the server and running in the client device, wherein the client device and the server communicate with each other via communications modules, through a network (e.g., application 222, communication modules 218, and network 150). Moreover, the application may include commands stored in a first memory circuit, the server may host the application via a coding engine including instructions stored in a second memory circuit, and the client device and server may store data in, and retrieve data from, a database (e.g., memory circuits 220, coding engine 230, and databases 152 or 252). The instructions in the memory circuits may be executed by processor circuits to cause the client device and the server to perform at least partially one or more of the steps in method 900 (e.g., processor circuits 212). In some embodiments, the coding engine includes a correlating tool and an NLP tool having a neural network algorithm or any other linear (e.g., multi-linear regression algorithm, and the like, cf. correlating tool 232, NLP tool 234, neural network 242, and machine learning 244) or non-linear algorithm such as a machine learning or artificial intelligence algorithm, to correlate a user textual feedback with medical codes according to technical rules and a code list (e.g., technical rules 236 and code list 238). Methods consistent with the present disclosure may include at least one step from method 900, and one or more steps in method 900 performed in a different order, overlapping in time, simultaneously, quasi-simultaneously, or at least partially overlapping in time.

Step 902 includes receiving a string input from a client device, the string input being part of a technical chart for a user of the client device, wherein the user is associated with a healthcare provider (e.g., a medical practitioner, a medical provider, a medical coder, an administrator, and the like) and the string input comprises a healthcare report of a patient. In some embodiments, step 902 includes opening a session for the user in a network portal hosted by a remote server, receiving the string input from the client device and causing a display to display the code occur within the session for the user.

Step 904 includes standardizing the string input according to a set of technical rules. In some embodiments, step 904 includes converting all the relevant fields in the EHR to a format that a model can process through the API (e.g., the coding engine and the tools therein). In some embodiments, different EHRs may provide different data formats. Accordingly, step 904 allows the same models (e.g., the coding engine and tools therein) to work seamlessly across a spectrum of EHRs without having to build a separate model for each individual EHR. In some embodiments, step 904 includes parsing the string input to identify at least one of a disease, a sign, or a symptom indicative of a patient's condition. In some embodiments, step 904 includes parsing the string input to identify a service provided by a healthcare professional to a patient.

Step 906 includes storing the string input in a database.

Step 908 includes providing at least a portion of the string input to a processing circuit.

Step 910 includes receiving, from the processing circuit, a code associated to the string input. In some embodiments, step 910 includes receiving multiple codes associated to the string input, and for each of the multiple codes receiving a matching factor between the code and the string input. In some embodiments, step 910 includes scoring the predictions by a confidence level and displaying the codes in a ranking order according to the score (e.g., the most confident codes first, and so on).

Step 912 includes causing a display in the client device to display the code for the user. In some embodiments, step 912 includes causing the display in the client device to display, to the user, a feedback to modify the string input. In some embodiments, step 912 includes linking a sidebar graphics indicative of the code to a portal display of a network application running in the client device. In some embodiments, step 912 includes determining a medication for the patient based on the healthcare report. In some embodiments, step 912 includes predicting an outcome for the patient based on the code associated with the string input. In some embodiments, step 912 includes diagnosing a patient's disease based on the code, storing the code in the database, and modifying a model in a memory circuit based on a correlation between the code and the portion of the string input, the model including instructions executed by the processing circuit for providing the code based on the string input.

Step 914 includes causing the display in the client device to display, to the user, a feedback for the string input.

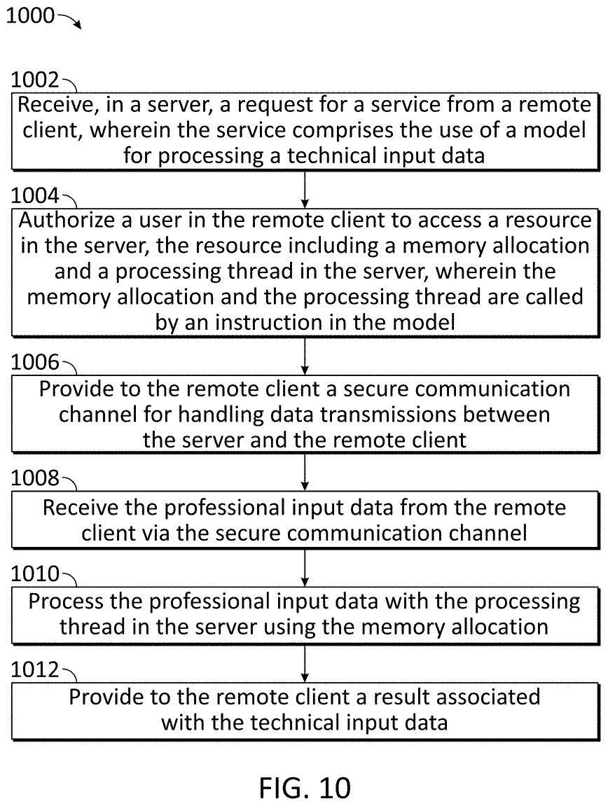

FIG. 10 illustrates steps in a method 1000 for providing a machine learning model to a remote client, according to some embodiments. In some embodiments, at least one or more of the steps in method 1000 may be performed by one or more devices such as a client device or a server in an architecture as disclosed herein (cf. client devices 110 and servers 130 in system architecture 100). Accordingly, in some embodiments, at least one or more of the steps in method 1000 may be performed by an application hosted by the server and running in the client device, wherein the client device and the server communicate with each other via communications modules, through a network (e.g., application 222, communication modules 218, and network 150). Moreover, the application may include commands stored in a first memory circuit, the server may host the application via a coding engine including instructions stored in a second memory circuit, and the client device and server may store data in, and retrieve data from, a database (e.g., memory circuits 220, coding engine 230, and databases 152 or 252). The instructions in the memory circuits may be executed by processor circuits to cause the client device and the server to perform at least partially one or more of the steps in method 1000 (e.g., processor circuits 212). In some embodiments, the coding engine includes a correlating tool and an NLP tool having a neural network algorithm or any other linear (e.g., multi-linear regression algorithm, and the like, cf. correlating tool 232, NLP tool 234, neural network 242, and machine learning 244) or non-linear algorithm such as a machine learning or artificial intelligence algorithm, to correlate a user textual feedback with medical codes according to technical rules and a code list (e.g., technical rules 236 and code list 238). Methods consistent with the present disclosure may include at least one step from method 1000, and one or more steps in method 1000 performed in a different order, overlapping in time, simultaneously, quasi-simultaneously, or at least partially overlapping in time.

Step 1002 includes receiving, in a server, a request for a service from a remote client, wherein the service comprises an access to a model that processes a technical input data. In some embodiments, the model that processes the technical input data includes a factor in a nonlinear operation, and step 1002 includes modifying the factor based on a comparison between the result associated with the technical input data and a known result stored in a database. In some embodiments, step 1002 includes providing, with the model that processes the technical input data, a confidence level for the result associated with the technical input data.

Step 1004 includes authorizing a user in the remote client to access a resource in the server, the resource including a memory allocation and a processing thread in the server, wherein the memory allocation and the processing thread are called by an instruction in the model. The user may include medical providers, medical coders, administrators, or any other professional associated with a healthcare provider. In some embodiments, step 1004 includes providing a path to the memory allocation to the remote client when the service is initiated. In some embodiments, step 1004 includes securing the memory allocation and the processing thread from access by a third party different from the remote client. In some embodiments, step 1004 includes authorizing a second user in a second remote client to access the resource in the server, the resource including the memory allocation and a second processing thread in the server. In some embodiments, step 1004 includes granting ‘read’ commands from the remote client in the processing thread. In some embodiments, step 1004 includes blocking ‘write’ commands from the remote client in the processing thread.

Step 1006 includes providing to the remote client a secure communication channel for handling data transmissions between the server and the remote client.

Step 1008 includes receiving the technical input data from the remote client via the secure communication channel.

Step 1010 includes processing the technical input data with the processing thread in the server using the memory allocation.

Step 1012 includes providing to the remote client a result associated with the technical input data. In some embodiments, step 1012 includes receiving, from the remote client, an outcome from the result and modifying the instruction in the model based on the outcome. In some embodiments, step 1012 includes erasing a data in the memory allocation when the service is terminated.

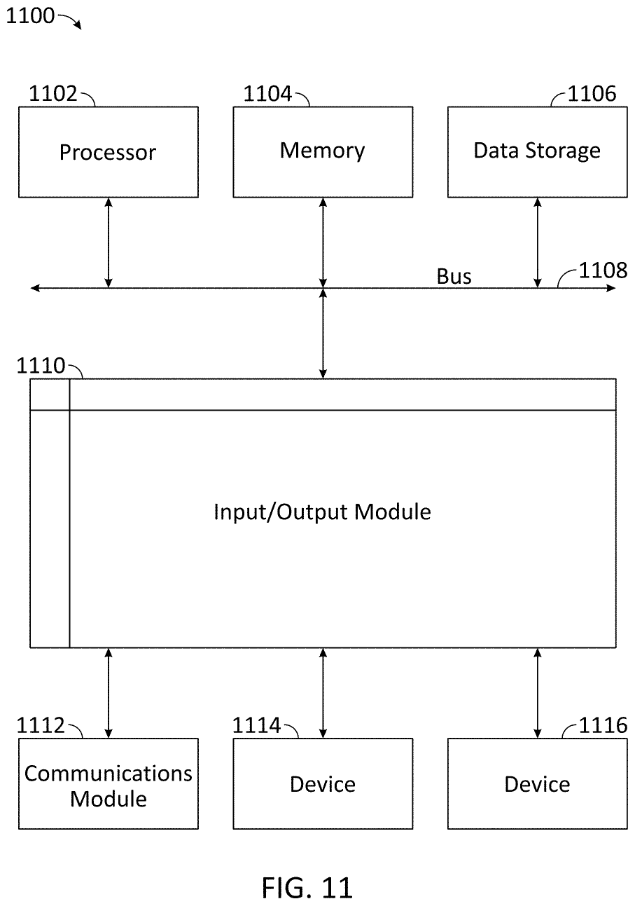

FIG. 11 illustrates a computer system 1100 for use in an architecture as disclosed herein, according to some embodiments. In certain aspects, the computer system 1100 may be implemented using hardware or a combination of software and hardware, either in a dedicated server, or integrated into another entity, or distributed across multiple entities.

Computer system 1100 includes a bus 1108 or other communication mechanism for communicating information, and a processor circuit 1102 coupled with bus 1108 for processing information. By way of example, the computer system 1100 may be implemented with one or more processor circuits 1102. Processor circuit 1102 may be a general-purpose microprocessor circuit, a microcontroller, a Digital Signal Processor circuit (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1100 can include, in addition to hardware, code that creates an execution environment for the computer program in question, e.g., code that constitutes processor circuit firmware, a protocol stack, a database management system, an operating system, or a combination of one or more of them stored in an included memory circuit, such as a Random Access Memory circuit (RAM), a flash memory circuit, a Read-Only Memory circuit (ROM), a Programmable Read-Only Memory circuit (PROM), an Erasable PROM (EPROM), registers, a hard disk, a removable disk, a CD-ROM, a DVD, or any other suitable storage device, coupled to bus 1108 for storing information and instructions to be executed by processor circuit 1102. The processor circuit 1102 and the memory circuit 1104 can be supplemented by, or incorporated in, special purpose logic circuitry.

The instructions may be stored in the memory circuit 1104 and implemented in one or more computer program products, i.e., one or more modules of computer program instructions encoded on a computer-readable medium for execution by, or to control the operation of, the computer system 1100, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory circuit 1104 may also be used for storing temporary variable or other intermediate information during execution of instructions to be executed by processor circuit 1102.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processor circuits executing one or more computer programs to perform functions by operating on input data and generating output.

Computer system 1100 further includes a data storage device 1106 such as a magnetic disk or optical disk, coupled to bus 1108 for storing information and instructions. Computer system 1100 may be coupled via input/output module 1110 to various devices. The input/output module 1110 can be any input/output module. Exemplary input/output modules 1110 include data ports such as USB ports. The input/output module 1110 is configured to connect to a communications module 1112. Exemplary communications modules 1112 include networking interface cards, such as Ethernet cards and modems. In certain aspects, the input/output module 1110 is configured to connect to a plurality of devices, such as an input device 1114 and/or an output device 1116. Exemplary input devices 1114 include a keyboard and a pointing device (e.g., a mouse or a trackball), by which a user can provide input to the computer system 1100. Other kinds of input devices 1114 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 1116 include display devices, such as an LCD (liquid crystal display) or light-emitting diode (LED) display, for displaying information to the user.

According to one aspect of the present disclosure, user device 110, try-on servers 130, and/or clinic 140 can be implemented using a computer system 1100 in response to processor circuit 1102 executing one or more sequences of one or more instructions contained in memory circuit 1104. Such instructions may be read into memory circuit 1104 from another machine-readable medium, such as data storage device 1106. Execution of the sequences of instructions contained in main memory circuit 1104 cause processor circuit 1102 to perform the process steps described herein. One or more processor circuits in a multi-processing arrangement may also be employed to execute the sequences of instructions contained in memory circuit 1104. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component (e.g., as a data server), or that includes a middleware component (e.g., an application server), or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication (e.g., a communication network). The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1100 can include clients and servers. A client and server are generally remote from each other and typically interact through a communication network. The relationship of client and server arises by virtue of computer programs running on the respective computers and having a client-server relationship to each other. Computer system 1100 can be, for example, and without limitation, a desktop computer, laptop computer, or tablet computer. Computer system 1100 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor circuit 1102 for execution. Such a medium may take many forms, including, but not limited to, non-volatile media, volatile media, and transmission media. Non-volatile media include, for example, optical or magnetic disks, such as data storage device 1106. Volatile media include dynamic memory circuit, such as memory circuit 1104. Transmission media include coaxial cables, copper wire, and fiber optics, including the wires that comprise bus 1108. Common forms of machine-readable media include, for example, a floppy disk, a flexible disk, a hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory circuit chip or cartridge, or any other medium from which a computer can read. The machine-readable storage medium can be a machine-readable storage device, a machine-readable storage substrate, a memory circuit device, a composition of matter effecting a machine-readable propagated signal, or a combination of one or more of them.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the term “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products. Other variations are within the scope of the following claims.

What is claimed is:

1. A computer-implemented method, comprising:
receiving a string input from a client device, the string input being part of a technical chart for a user of the client device, wherein the user is associated with a healthcare provider and the string input comprises a healthcare report of a patient;
standardizing the string input according to a set of technical rules;
storing the string input in a database;
providing at least a portion of the string input to a processing circuit;
receiving, from the processing circuit, a code associated with the string input;
causing a display in the client device to display the code for the user; and
providing the code to an electronic healthcare record including the healthcare report, upon selection by the user.

2. The computer-implemented method of claim 1, further comprising opening a session for the user in a network portal hosted by a remote server, wherein the receiving the string input from the client device and causing a display to display the code occur within the session for the user.

3. The computer-implemented method of claim 1, wherein standardizing the string input according to a set of technical rules comprises converting one or more fields in an electronic health record to a format readable by the processing circuit.

4. The computer-implemented method of claim 1, wherein standardizing the string input according to a set of technical rules comprises parsing the string input to identify at least one of a disease, a sign or a symptom indicative of a patient's condition.

5. The computer-implemented method of claim 1, wherein standardizing the string input according to a set of technical rules comprises parsing the string input to identify a service provided by a healthcare professional to a patient.

6. The computer-implemented method of claim 1, wherein receiving a code associated with the string input comprises receiving multiple codes associated to the string input, and for each of the multiple codes receiving a matching factor between the code and the string input.

7. The computer-implemented method of claim 1, wherein receiving a code associated with the string input comprises:
receiving a code indicative of a level of evaluation and management associated with the string input; and
receiving a code indicative of a disease or symptom associated with the string input.

8. The computer-implemented method of claim 1, wherein the string input comprises a therapy applied to a patient by a healthcare practitioner and receiving a code associated with the string input comprises receiving a procedure terminology code.

9. The computer-implemented method of claim 1, further comprising:
receiving a clinical documentation improvement suggestion from the processing circuit, and causing the display in the client device to display the clinical documentation improvement suggestion;
causing the display in the client device to load a list of prior interaction items for a user of the client device, and enabling the user to select to continue at least one prior interaction item; and
causing the display in the client device to display, to the user, a feedback to modify the string input.

10. The computer-implemented method of claim 1, wherein causing the display in the client device to display the code for the user comprises:
linking a sidebar graphics indicative of the code to a portal display of a network application running in the client device; and
ranking multiple codes in the display according to a confidence level score.

11. A computer-implemented method, comprising:
receiving, in a server, a request for a service from a remote client, wherein the service comprises an access to a model that processes a technical input data;
authorizing a user in the remote client to access a resource in the server, the resource including a memory allocation and a processing thread in the server, wherein the memory allocation and the processing thread are called by an instruction in the model;
providing to the remote client a secure communication channel for handling data transmissions between the server and the remote client;
receiving the technical input data from the remote client via the secure communication channel;
processing the technical input data with the processing thread in the server using the memory allocation; and
providing, to the remote client, a result associated with the technical input data.

12. The computer-implemented method of claim 11, wherein the model that processes the technical input data comprises a factor in a nonlinear operation, further comprising modifying the factor based on a comparison between the result associated with the technical input data and a known result stored in a database.

13. The computer-implemented method of claim 11, further comprising providing, with the model that processes the technical input data, a confidence level for the result associated with the technical input data.

14. The computer-implemented method of claim 11, further comprising providing, to the remote client, a path to the memory allocation when the service is initiated.

15. The computer-implemented method of claim 11, further comprising:
securing the memory allocation and the processing thread from access by a third party different from the remote client;
authorizing a second user in a second remote client to access the resource in the server, the resource including the memory allocation and a second processing thread in the server;
granting ‘read’ commands from the remote client in the processing thread; and
blocking ‘write’ command from the remote client in the processing thread.

16. The computer-implemented method of claim 11, further comprising:
receiving, from the remote client, an outcome from the result and modifying the instruction in the model based on the outcome; and
erasing a data in the memory allocation when the service is terminated.

17. The computer-implemented method of claim 11, wherein the result is a diagnosis code and authorizing a user to access the resource in the server comprises calling an application programming interface in a diagnosis model engine to find the diagnosis code.

18. The computer-implemented method of claim 11, wherein the result is a procedure code and authorizing a user to access the resource in the server comprises calling an application programming interface in a procedure model engine to find the procedure code.

19. The computer-implemented method of claim 11, wherein the result is an evaluation and management code and authorizing a user to access the resource in the server comprises calling an application programming interface in an evaluation and management engine to find the evaluation and management code.

20. The computer-implemented method of claim 11, result includes a recommendation to improve a clinical documentation for a healthcare provider, and providing the result comprises matching the technical input data with a set of technical rules associated with a condition of a patient treated by the healthcare provider.
